(19) United States
(12) Patent Application Publication
(10) Pub. No.: US 2024/0386584 A1
(43) Pub. Date: Nov. 21, 2024

(54) SYSTEM AND METHOD FOR IDENTIFYING A REGION OF INTEREST

(71) Applicant: Brainlab AG, Munich (DE)

(72) Inventors: Wei WEI, Munich (DE); Daniel MENA, Munich (DE)

(21) Appl. No.: 18/693,772

(22) PCT Filed: Sep. 29, 2021
(86) PCT No.: PCT/EP2021/076747

§ 371 Date: Mar. 20, 2024

(51) Int. Cl. G06T 7/33; A61B 8/00; G06T 7/13; G06V 10/25; G06V 20/50

(52) U.S. Cl. CPC G06T 7/337; G06T 7/13; G06V 10/25; G06V 20/50; A61B 8/42; G06T 2200/04; G06T 2207/10081; G06T 2207/10088; G06T 2207/10136; G06V 2201/03

(57) ABSTRACT

A system and a computer-implemented method for identifying a region of interest for image registration is provided. The method includes analyzing three-dimensional CT or MRI image data of a tissue to identify one or more structural elements therein and determining a spatial arrangement of the structural elements, accessing first ultrasound image data of the tissue and performing a first image registration of the first ultrasound image data with the CT or MRI image data at a first accuracy, and identifying, based on the spatial arrangement of the structural elements, a region of interest for a subsequent second image registration of second ultrasound image data of the tissue with the CT or MRI image data at a second accuracy that is higher than the first accuracy.

### FIELD OF THE INVENTION

The present invention relates to a computer-implemented method and a system for identifying a region of interest for image registration, a corresponding computer program product and computer-readable medium.

### TECHNICAL BACKGROUND

In the setting of imaging of tissues, a very accurate method for imaging is imaging by means of CT or MRI. In particular, three-dimensional CT or MRI image data provide high-accuracy representations of the tissue.

However, interactions with tissues often may involve guiding or navigating instruments supported by image data obtained by performing imaging of the tissue. However, for various reasons, the tissue may move over time during the interaction with the tissue. Accordingly, any static image registration done beforehand will be invalid. This means that the initially determined image data position cannot, as such, be used reliably throughout the process of interacting with the tissue, particularly for steps of the interaction with the tissue that require guiding and/or navigating instruments by means of image data. However, monitoring the tissue by means of CT or MRI over an extended period of time may be cumbersome, impede the interaction with tissue, or may not be feasible at all.

Ultrasound imaging provides an imaging method that, in principle, can be more easily used throughout the interaction to acquire real-time data. It is generally not harmful to a user performing the interaction or to the tissue that is being analysed and ultrasound probes are easy to handle and allow for increased flexibility.

However, ultrasound images generally have lower image quality than CT or MRI images, which may be detrimental to for proper guidance or navigation of instruments.

Generally, a registration of ultrasound images and with CT or MRI images to overcome this drawback.

For example, co-aligning initial three-dimensional CT or MRI image data and two-dimensional ultrasound image data may an option to overcome this drawback. However, for registration, generally some structural elements, for example vessel structures, in the tissues are mapped onto each other. In the two-dimensional ultrasound image data, inherently, these structural elements are only partially visible, as only one plane is depicted and the field of view is limited. Accordingly, achieving high accuracy and robustness of the registration is difficult.

Alternatively, three-dimensional ultrasound image data may be mapped onto initial three-dimensional CT or MRI image data. Some of the above-mentioned drawbacks are overcome, as the field of view is increased and structural elements are more visible, which allows for higher accuracy and robustness. However, the transducer needs to acquire a large amount of signal data and the processing of the signal data to reconstruct an image volume and perform the registration requires a large amount of computing resources. This may lead to a time-delay in the reconstruction of the image that may not be desirable, for example, when relying on the images for guiding or navigating instruments.

Thus, to summarize, available methods for image registration require a large amount of computing resources, particularly when three-dimensional ultrasound image data are used, and/or do not provide the required registration accuracy, particularly when two-dimensional ultrasound image data are used.

It is an object of the present invention to provide a method that overcomes these drawbacks.

The present invention can be used, for example, by being incorporated in surgical navigation systems. Examples for such navigation systems are Curve® Navigation or Kick® Navigation, but can also be used for various other purposes than surgical navigation systems, e.g., in any systems that involve obtaining positional data from ultrasound images to improve their accuracy.

Aspects of the present invention, examples and exemplary steps and their embodiments are disclosed in the following. Different exemplary features of the invention can be combined in accordance with the invention wherever technically expedient and feasible.

### EXEMPLARY SHORT DESCRIPTION OF THE INVENTION

In the following, a short description of the specific features of the present invention is given which shall not be understood to limit the invention only to the features or a combination of the features described in this section.

A system and a computer-implemented method for identifying a region of interest for image registration is provided. The method comprises analyzing three-dimensional CT or MRI image data of a tissue to identify one or more structural elements therein and determining a spatial arrangement of the structural elements, accessing first ultrasound image data of the tissue and performing a first image registration of the first ultrasound image data with the CT or MRI image data at a first accuracy, and identifying, based on the spatial arrangement of the structural elements, a region of interest for a subsequent second image registration of second ultrasound image data of the tissue with the CT or MRI image data at a second accuracy that is higher than the first accuracy.

### GENERAL DESCRIPTION OF THE INVENTION

In this section, a description of the general features of the present invention is given for example by referring to possible embodiments of the invention.

A system and a computer-implemented method for identifying a region of interest for image registration is provided. The method comprises analyzing three-dimensional CT or MRI image data of a tissue to identify one or more structural elements therein and determining a spatial arrangement of the structural elements. The method also comprises accessing first ultrasound image data of the tissue and performing a first image registration of the first ultrasound image data with the CT or MRI image data at a first accuracy. The method further comprises identifying, based on the spatial arrangement of the structural elements, a region of interest for a subsequent second image registration of second ultrasound image data of the tissue with the CT or MRI image data at a second accuracy that is higher than the first accuracy.

As already mentioned above, CT or MRI image data provides high accuracy. Accordingly, the tissue's structure, for example structural elements thereof, like vessels, can be derived in a reliable manner from the CT or MRI image data.

Structural elements provide a very good basis for performing high-accuracy image registration. Accordingly, identifying a region of interest that, based on the arrangement of the structural elements, is suitable for performing high-accuracy image registration is advantageous because image data for the region of interest may then be used as a basis for subsequent (second) image registration steps.

The first image registration step, performed at lower accuracy, is used to allow for a reliable, yet relatively fast and resource-saving, mapping of the (first) ultrasound image data to be used in identifying the region of interest. For example, the low accuracy may be due to using an image registration that puts more weight on speed than on accuracy or by using a low amount of information, e.g., low pixel or voxel density or low number of structural elements.

Based thereon, the region of interest, which may be used in optional subsequent steps for a second image registration, may be determined. Aspects concerning the region of interest are explained in more detail further below. For performing the subsequent steps, additional ultrasound image data may be sampled for the region of interest and/or additional ultrasound image data of the region of interest that had already been sampled, yet disregarded for the first image registration, may be taken into account for the second image registration to provide more detailed data in the region of interest.

Thus, it will be understood that at least some of the drawbacks of available methods for image registration, for example that they require a large amount of computing resources, particularly when three-dimensional ultrasound image data are used, and/or do not provide the required registration accuracy, particularly when two-dimensional ultrasound image data are used, are overcome by the method of the present disclosure.

Structural elements, in this disclosure, may, for example, refer to structures of the tissue, for example vessels, but also borders of organs within the tissue, e.g., borders of a liver or a gallbladder, or other anatomical structures within the tissue.

Accessing image data, according to the present disclosure, may be an example of the more broadly defined acquiring of data explained below. Specifically, accessing data might not involve the actual step measurement of the image data by means of scanners or transducers.

The method of this disclosure may provide a transformation of structural elements, for example of a vessel tree, in the CT or MRI image data, which structural elements are used in the registration, to the ultrasound volume.

It can be understood from the above, that the method of this disclosure provides a registration in a coarse-to-fine fashion.

The accuracy of the image registration can be influenced by properties of the images, e.g., resolution. Alternatively, or in addition, it may depend on settings of the processing device, particularly an optimizer involved in the image registration.

Such settings may further include, for example, settings having an influence on speed, robustness and accuracy with which the processing device is to perform the image registration.

Such settings can be termination criteria for the optimizer (e.g. max. number of iterations, similarity change, threshold for similarity or similarity change).

It is noted that any methods known in the art for image registration may be employed for the first image registration, including landmark-based techniques, intensity-based techniques, techniques using segmentation and/or atlas data, which are explained in more detail below.

Furthermore, it is noted that the identification of the region of interest may be fully automated. In some cases, the fully automated identification may be based, for example, on predetermined calculations, predetermined constraints and/or predetermined boundary conditions. Alternatively, or in particular, deep learning may be used in implementing fully automated identification. Boundary conditions may include that the region of interest be a plane with certain properties and/or that the region of interest be a, particularly contiguous, assembly of voxels with certain properties.

Alternatively, the identification may be semi-automatic, i.e., the identification may involve user interaction. For example, based on predetermined calculations, predetermined constraints and/or predetermined boundary conditions, a region of interest may be suggested to the user and the user may confirm, by means of user input, said proposed region of interest or modify the suggested region of interest, in which case the modified region of interest may be identified as the region of interest. Alternatively or in addition, based on predetermined calculations, predetermined constraints and/or predetermined boundary conditions, a region of interest may be suggested to the user and it may be inferred from user behavior whether the region of interest is confirmed or modified, for example, if the user re-positions the ultrasound probe in a particular manner.

Identifying the region of interest based on the spatial arrangement of the structural elements may comprise determining the number and/or density of the structural elements of different areas in the CT or MRI image data.

For example, the method may identify a region as the region of interest in response to determining that it has more than a predetermined threshold of structural elements and/or that the density of structural elements in the region is above a predetermined threshold. Alternatively, or in addition, the method may identify a certain region as the region of interest in response to determining that the number and/or density of structural elements exceeds the number and/or density of structural elements in other areas by a predetermined amount.

Using the number and/or density of structural elements for identifying the region of interest allows for increasing the accuracy of a second image registration, as this accuracy may increase with an increasing number of structural elements being present in the region of interest.

Alternatively, or in addition, identifying the region of interest based on the spatial arrangement of the structural elements may comprise determining areas in the CT or MRI image data that do not have any structural elements. In particular, identifying the region of interest may comprise identifying only areas wherein at least one structural element is present as being part of the region of interest.

In particular, the method may comprise, in response to determining that an area in the CT or MRI image data does not have any structural elements, determine that this area is not part of the region of interest. Accordingly, it may be possible to quickly determine areas that are not particularly suitable for improving accuracy.

The above feature allows for a quick process of eliminating some areas from candidates for the region of interest. For example, such an area may be seen as an area of no interest, e.g. an avoidance region or exclusion region.

Identifying the region of interest based on the spatial arrangement of the structural elements may be performed taking into account boundary conditions as to the region of interest.

In particular, this may allow for improved automation of identifying the region of interest. The boundary conditions may be accessible in a computer-readable manner and used for one or more automatically performed steps comprised in identifying the region of interest, e.g., for use by a processing unit. Even if the region of interest is not identified in a fully automated manner, any suggestions made to a user for user selection or modification, will be improved with boundary conditions being taken into account for the suggestion.

As an example, size and/or shape and/or orientation and/or position of the region of interest may be taken into account as boundary conditions. Alternatively, or in addition, boundary conditions like limits for computing resource consumption and/or processed data volume and/or expected computation times respectively required for the second image registration may be taken into account as boundary conditions.

These conditions allow for improving the identification of the region of interest in terms of the technical requirements at hand and/or the conditions in which the method is performed.

Performing the first image registration may comprise aligning at least some of the structural elements identified in the CT or MRI image data with corresponding structural elements in the first ultrasound image data.

The aligning of the first image registration can be performed using any suitable method known in the art. The aligning may be performed in such a manner that alignment speed is given a higher weight than alignment accuracy. Thus, the alignment may lead to a lower resolution registration. As an example for the alignment, the structural elements that are aligned may comprise one or more tubular structures, in particular one or more vessels or vessel trees in the tissue.

Performing the second image registration may comprise aligning at least some of the structural elements identified in the CT or MRI image data in the region of interest with corresponding structural elements in the second ultrasound image data.

The aligning of the second image registration can be performed using any suitable method known in the art. The aligning may be performed in such a manner that alignment accuracy is given a higher weight than alignment speed. The alignment accuracy during the second image registration may be higher than alignment accuracy during the first image registration. Thus, the alignment may lead to a higher resolution registration. As an example for the alignment, the structural elements that are aligned may comprise one or more tubular structures, in particular one or more vessels or vessel trees in the tissue.

Identifying one or more structural elements may comprise determining high order image derivatives in the CT or MRI image data and determining, based thereon, the presence and approximate shape and/or orientation of the one or more structural elements.

The use of higher order derivatives of the three-dimensional CT or MRI image data allows for a reliable identification of structural elements. In particular, tubular elements can be identified with high reliability. As a consequence of higher reliability of identification of the structural elements, the identification of the region of interest is also more reliable.

As a non-limiting illustrative example, a Hessian Matrix H (I) may be obtained for each voxel of the three-dimensional CT or MRI image data.

Identifying the structural elements may comprise obtaining the Eigenvalues of a Hessian matrix applied to the CT or MRI image data and, based on the Eigenvalues, identifying the structural elements.

In particular, when performing a singular value decomposition, Eigenvalues and Eigenvectors may be obtained. They may serve as shape descriptors.

For example, different combinations of Eigenvalues may indicate the presence of plate-like, tubular, or blob-like structures, or may indicated that the data is noisy or no preferred direction is detected.

As already mentioned above, the method may comprise identifying tubular structures as structural elements. In a tissue, there may be a plurality of vessels that are generally tubular in shape. They can be reliably identified and, therefore, are particularly suitable as a basis for selection of the region of interest and also for performing image registration at high accuracy. It is conceivable to focus on and/or filter for tubular structures for the image registration. This could further improve registration accuracy.

In particular, identifying the structural elements may comprise categorizing each of the Eigenvalues as low or as high positive or as high negative and determining that a structural element is present when a predetermined combination of categories of the Eigenvalues is determined.

This type of determining that a structural element is present is particularly advantageous as the categorization is simple and efficient, such that the computation effort is low, while the determination still yields sufficiently good results for reliably determining the region of interest. For example, for reliably determining the region of interest, it may not be necessary to obtain a very precise categorization of the structural elements, as long as, for example, approximately tubular shaped elements can be identified.

The categorization may be performed, for example, by comparing the Eigenvalues to predetermined thresholds that define whether an Eigenvalue is low, high positive, or high negative.

The structural elements may, for example, comprise a tubular structure and the method may comprise identifying that the tubular structure is present when a first Eigenvalue is categorized as low and a second Eigenvalue and a third Eigenvalue are each categorized as high positive or when a first Eigenvalue is categorized as low and a second Eigenvalue and a third Eigenvalue are each categorized as high negative.

Although tubular structures are mentioned as an example above, other types of structures can also be determined on the basis of the Eigenvalues. For example, plate-like structures can be present when a low first and second Eigenvalue are low and a third Eigenvalue is high positive or high negative. A blob-like structure may be present when all three Eigenvalues are high positive or all three Eigenvalues are high negative.

The first ultrasound image data and the second ultrasound image data may be three-dimensional ultrasound image data, particularly acquired by a 3D transducer. The first ultrasound image data may comprise a first subset of voxels of ultrasound image data of the tissue, particularly of evenly distributed voxels. The second ultrasound image data may comprise a second subset of voxels of ultrasound image data of the tissue, the second subset of voxels having a higher voxel-density in the region of interest than the first subset of voxels. In particular, the second subset of voxels may comprise at least part of the first subset of voxels.

Throughout the present disclosure, when a 3D transducer is disclosed, said 3D transducer may be a real-time or near real-time 3D transducer, for example a 2D array transducer, e.g. comprising a matrix array or row column array, or a mechanical 3D probe, also referred to as a wobbler.

In other words, the voxel-density in the second image data may be non-uniform.

When performing a second image registration using the second ultrasound image data, a higher voxel-density may be used for the image registration in the region of interest than in the other regions.

As already mentioned above, using three-dimensional ultrasound image data, in general allows for high accuracy. However, as also mentioned above, the amount of computing resources required for obtaining and registering three-dimensional image data is also high.

The above, accordingly, allows for ensuring high accuracy of the second image registration by having a high voxel-density in the region of interest, where more structural elements may be present, while at the same time reducing the amount of computing resources that are required by using a low voxel-density where fewer structural elements are present, i.e., where a high voxel-density would barely improve the accuracy of the image registration.

The first ultrasound image data and the second ultrasound image data may be three-dimensional ultrasound image data, particularly acquired by a/the 3D transducer, and identifying the region of interest may comprise identifying only areas wherein at least one of the structural elements, in particular a/the tubular structure, is present as being part of the region of interest.

Thus, while ensuring high accuracy, the amount of computing resources can be reduced significantly.

The first ultrasound image data and the second ultrasound image data may be three-dimensional ultrasound image data, particularly acquired by a/the 3D transducer, and the second ultrasound image data may provide more detail in the region of interest than the first image data, particularly may have a higher voxel-density in the region of interest than the first image data.

Alternatively, or in addition, the first ultrasound image data and the second ultrasound image data may be three-dimensional ultrasound image data, particularly acquired by a/the 3D transducer, and the second ultrasound image data may be non-uniform, providing more detail in the region of interest than in other regions, particularly may have a higher voxel-density in the region of interest than in other regions.

Thus, while ensuring high accuracy, the amount of computing resources can be reduced. As briefly mentioned above, higher voxel-density may, in particular, be used in areas with a higher number or density of structural elements, for example tubular structures like vessels, than in other areas. As such, the overall amount of resources for a second image registration is reduced compared to the case of uniformly high voxel-density without considerably reducing the accuracy, which is mostly ensured by the mapping of the structural elements. Accordingly, the processing time and, accordingly, time to reconstruct images, will also be decreased.

The first ultrasound image data may be two-dimensional ultrasound image data obtained in a first plane and the second ultrasound image data may be two-dimensional image data to be obtained in a second plane.

The second plane or part of the second plane may comprise the region of interest.

Determining the region of interest may comprise determining the second plane by performing an optimization procedure that optimizes for the first plane and the second plane being close to perpendicular to each other and for the second plane comprising a high number and/or density of the structural elements.

The determining of the second plane may be performed in such a manner as to obtain an optimal plane constrained by the conditions that the second plane comprise as many structural elements, e.g. tubular structures, as possible and that the second plane be perpendicular or as close as possible to perpendicular to the first plane.

The more structural elements are comprised in the image data, the better the accuracy of image registration based on the structural elements. The closer the second plane is to being perpendicular to the first plane, the more additional information is gained from image data obtained in the second plane. Thus, determining the second plane accordingly allows for improving accuracy of the registration significantly.

A non-limiting illustrative example for such an optimization is shown below. In this example, the so-called Vesselness response is used as an example for the optimizing for high number and/or density of the structural elements. As will be seen below, the Eigenvalues of the three-dimensional CT or MRI image data may be used for determining the Vesselness response. The plane can be determined using the following equation:

$$Arg {max}_{( \vec{{n}_{2}} , {P}_{2} )} ( \frac{{k}_{1} {\sum }_{σ}^{N} ϑ ( σ )}{N} + {k}_{2} ‖ \vec{{n}_{1}} ⊗ \vec{{n}_{2}} ‖ )$$

with the following parameters:
\vec{n₁} Normal vector of the first plane
\vec{n₂} Normal vector of the second plane
P₂ 3D Point to define the second plane
σ 3D point on the plane constrained by

$$\frac{( σ - {P}_{2} )}{\vert ( σ - {P}_{2} ) \vert} ⊗ \vec{{n}_{2}} = 0$$

N Collection of all points on the second plane
k₁ Weight coefficient for vesselness term
k₂ Weight coefficient for perpendicular plane constraint
ϑ(σ) Vesselness response on point σ, wherein

$$ϑ ( σ ) = \{ \begin{matrix}0 \\ ( 1 - {e}^{- \frac{{R}_{A}^{2}}{2 \cdot {α}^{2}}} ) ( {e}^{- \frac{{R}_{B}^{2}}{2 \cdot {β}^{2}}} ) ( 1 - {e}^{- \frac{{S}^{2}}{2 \cdot {c}^{2}}} )\end{matrix}$$

### if E2>0 and E3>0 Else

$${R}_{A} = \frac{\vert E 2 ( σ ) \vert}{\vert E 3 ( σ ) \vert} ,$$

$${R}_{B} = \frac{\vert E 1 ( σ ) \vert}{\sqrt{\vert E 2 ( σ ) E 3 ( σ ) \vert}} ,$$

$$S = \sqrt{E {1}^{2} + E {2}^{2} + E {3}^{2}}$$

In the above definition of the Vesselness response, α, β, c may be empirical or semi-empirical parameters, for example.

E1, E2, E3 are the first, second and third eigenvalues after applying single value decomposition to the three-dimensional CT or MRI image data, for example as described above.

It is noted that the above optimization is by way of example only. The method may instead provide one or more boundary conditions for the number of structural elements comprised in the second plane and optimize for the second plane to be perpendicular or as close as possible to perpendicular to the first plane or to provide one or more boundary conditions for the angle of the second plane with respect to the first plane and optimize for the second plane to comprise as many structural elements, e.g. tubular structures, as possible.

Any of the methods described above may further comprise performing the second image registration of the second ultrasound image data with the CT or MRI image data at the second accuracy. The second ultrasound image data may include ultrasound image data obtained in the second plane. In particular, the transducer used for obtaining the ultrasound image data may be automatically positioned, in response to the second plane being identified, so as to obtain image data in the second plane. Alternatively, or in addition, an output indicating how to position the transducer may be provided to a user, for example via a display and/or via other optical and/or via acoustic indicators providing guidance. Thus, the user may be guided to position the transducer in such a manner that image data is obtained in the second plane.

The image data used for the first image registration may also be used for the second image registration. This applies to both, the case where the first and second ultrasound image data are two-dimensional image data and the case where the first and second image data are three-dimensional image data.

As an example, when the first and second image data are two-dimensional ultrasound image data, the data obtained for the first plane need not be discarded, but may be used in addition to the image data from the second plane to provide even higher accuracy, particularly, if the image data from the first plane is registered with a higher weight on accuracy, rather than speed.

When the first and second image data are three-dimensional ultrasound image data, as an example, the second ultrasound image data may include the second subset of voxels of ultrasound image data of the tissue, the second subset of voxels having a higher voxel-density in the region of interest than the first subset of voxels.

The first ultrasound image data may be ultrasound image data obtained at a later time than the CT or MRI image data. In particular, the first ultrasound image data may be obtained, in particular continuously, over an extended period of time during interaction with the tissue. This may allow for improved monitoring of the interaction with the tissue and providing improved guidance and navigation during the interaction.

The second ultrasound image data may comprise at least part of the first ultrasound image data, particularly in case the ultrasound image data is three-dimensional image data. This means that at least part of the image data used already for the first image registration may also be used for the second image registration. This is advantageous in terms of accuracy and/or computing resources as compared to using entirely different image data.

The second ultrasound image data, in particular the ultrasound image data for the second plane, may be ultrasound image data obtained after identifying the region of interest. This reduces the overall time and effort required. In particular, obtaining data in several planes would require a significant amount of effort, which can be avoided if only data obtained in one plane is required at first and then only data obtained in one additional plane, i.e., the second plane, is required. Due to the above-described identification of the second plane, the accuracy can at the same time be ensured.

The second ultrasound image data may be ultrasound image data obtained before identifying the region of interest, in particular, ultrasound image data obtained together with the first ultrasound image data and/or ultrasound image data that at least partially comprises the first ultrasound image data.

The second image registration may be used for real-time adjustment of posture of an ultrasound transducer with respect to the tissue.

The method may comprise obtaining CT or MRI image data of the tissue using a CT or MRI scanner. Alternatively, or in addition, the method may comprise obtaining, particularly subsequently to obtaining CT or MRI image data, the first ultrasound image data and/or the second ultrasound image data using a two-dimensional or a three-dimensional ultrasound transducer.

In particular, the method may comprise obtaining the second ultrasound image data, or at least part of the second ultrasound image data, in response to identifying the region of interest.

Two-dimensional ultrasound image data may be obtained, particularly in the above-described first and/or second planes, by means of an x-shaped probe and/or an array. The probe may be a motorized probe and/or may be positioned by ways of user guidance.

The present disclosure also provides a system for identifying a region of interest for image registration, the system configured to perform any of the methods described in the present disclosure. In particular, the system may comprise a processing unit (which may also be referred to as a processor) that is configured to, in particular automatically, perform one or more of, in particular all, of the method steps described herein, unless specified otherwise, e.g., unless it is specified that the steps are performed by the user.

The system may further comprise a CT or MRI scanner configured to obtain the CT or MRI image data and/or an ultrasound transducer configured to obtain the first ultrasound image data and/or the second ultrasound image data.

The processing unit may be comprised in computer. The system, in particular the computer, may also comprise at least one memory unit or configured to access a memory unit. The memory unit may be comprised in the computer-readable medium according to the invention.

The present disclosure also provides a computer program product comprising instructions which, when the program is executed by a computer, cause the computer to carry out any of the methods described herein.

The present disclosure also provides a computer-readable medium comprising instructions which, when executed by a computer, cause the computer to carry out any of the methods described herein.

The present disclosure also provides use of a system as described above for determining a region of interest for image registration. In particular, the present disclosure provides the use of the system for guidance and/or navigation of an ultrasound probe and/or an instrument used for interacting with the tissue making use of the second image registration.

The features and advantages outlined above in the context of the method similarly apply to the system, the computer program product, and computer-readable medium, and use of the system described herein.

For example, the invention does not involve or in particular comprise or encompass an invasive step which would represent a substantial physical interference with the body requiring professional medical expertise to be carried out and entailing a substantial health risk even when carried out with the required professional care and expertise. For example, the invention does not comprise a step of positioning a medical implant in order to fasten it to an anatomical structure or a step of fastening the medical implant to the anatomical structure or a step of preparing the anatomical structure for having the medical implant fastened to it. More particularly, the invention does not involve or in particular comprise or encompass any surgical or therapeutic activity. The invention is instead directed as applicable to guidance and/or navigation of an ultrasound probe and/or an instrument used for interacting with the tissue. For this reason alone, no surgical or therapeutic activity and in particular no surgical or therapeutic step is necessitated or implied by carrying out the invention.

### DEFINITIONS

In this section, definitions for specific terminology used in this disclosure are offered which also form part of the present disclosure.

### Computer Implemented Method

The method in accordance with the invention is for example a computer implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n- and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

The invention also relates to a computer program product comprising instructions which, when the program is executed by (also referred to as running on) a computer, cause the computer to carry out or (also referred to as performing) one or more or all of the method steps described herein and/or to a computer-readable medium, also referred to as program storage medium, on which the program may be stored (in particular in a non-transitory form). Alternatively, or in addition, the present invention may relate to a computer comprising said program storage medium and/or to a (physical, for example electrical, for example technically generated) signal wave, for example a digital signal wave, carrying information which represents the program, for example the aforementioned program, which for example comprises code means which are adapted to perform any or all of the method steps described herein.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

### Acquiring Data

The expression “acquiring data” for example encompasses (within the framework of a computer implemented method) the scenario in which the data are determined by the computer implemented method or program. Determining data for example encompasses measuring physical quantities and transforming the measured values into data, for example digital data, and/or computing (and e.g. outputting) the data by means of a computer and for example within the framework of the method in accordance with the invention. The meaning of “acquiring data” also for example encompasses the scenario in which the data are received or retrieved by (e.g. input to) the computer implemented method or program, for example from another program, a previous method step or a data storage medium, for example for further processing by the computer implemented method or program. Generation of the data to be acquired may but need not be part of the method in accordance with the invention. The expression “acquiring data” can therefore also for example mean waiting to receive data and/or receiving the data. The received data can for example be inputted via an interface. The expression “acquiring data” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively, or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention.

The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

### Registering

The n-dimensional image of a tissue is registered when the spatial location of each point of an actual object within a space, for example one or more vessels in a tissue, is assigned an image data point of an image (CT, MR, etc.) stored in a navigation system.

### Image Registration

Image registration is the process of transforming different sets of data into one co-ordinate system. The data can be multiple photographs and/or data from different sensors, different times or different viewpoints. It is used in computer vision, medical imaging and in compiling and analyzing images and data from satellites. Registration is necessary in order to be able to compare or integrate the data obtained from these different measurements.

### Landmarks

A landmark is a defined element of an anatomical body part which is always identical or recurs with a high degree of similarity in the same anatomical body part of multiple patients. Typical landmarks are for example the epicondyles of a femoral bone or the tips of the transverse processes and/or dorsal process of a vertebra. The points (main points or auxiliary points) can represent such landmarks. A landmark which lies on (for example on the surface of) a characteristic anatomical structure of the body part can also represent said structure. The landmark can represent the anatomical structure as a whole or only a point or part of it. A landmark can also for example lie on the anatomical structure, which is for example a prominent structure. An example of such an anatomical structure is the posterior aspect of the iliac crest. Another example of a landmark is one defined by the rim of the acetabulum, for instance by the centre of said rim. In another example, a landmark represents the bottom or deepest point of an acetabulum, which is derived from a multitude of detection points. Thus, one landmark can for example represent a multitude of detection points. As mentioned above, a landmark can represent an anatomical characteristic which is defined on the basis of a characteristic structure of the body part. Additionally, a landmark can also represent an anatomical characteristic defined by a relative movement of two body parts, such as the rotational centre of the femur when moved relative to the acetabulum.

### Referencing

Determining the position is referred to as referencing if it implies informing a navigation system of said position in a reference system of the navigation system.

### Atlas/Atlas Segmentation

Preferably, atlas data is acquired which describes (for example defines, more particularly represents and/or is) a general three-dimensional shape of the anatomical body part. The atlas data therefore represents an atlas of the anatomical body part. An atlas typically consists of a plurality of generic models of objects, wherein the generic models of the objects together form a complex structure. For example, the atlas constitutes a statistical model of a body (for example, a part of the body) which has been generated from anatomic information gathered from a plurality of human bodies, for example from medical image data containing images of such human bodies. In principle, the atlas data therefore represents the result of a statistical analysis of such medical image data for a plurality of human bodies. This result can be output as an image—the atlas data therefore contains or is comparable to medical image data. Such a comparison can be carried out for example by applying an image fusion algorithm which conducts an image fusion between the atlas data and the medical image data. The result of the comparison can be a measure of similarity between the atlas data and the medical image data. The atlas data comprises image information (for example, positional image information) which can be matched (for example by applying an elastic or rigid image fusion algorithm) for example to image information (for example, positional image information) contained in medical image data so as to for example compare the atlas data to the medical image data in order to determine the position of anatomical structures in the medical image data which correspond to anatomical structures defined by the atlas data.

The human bodies, the anatomy of which serves as an input for generating the atlas data, advantageously share a common feature such as at least one of gender, age, ethnicity, body measurements (e.g. size and/or mass) and pathologic state. The anatomic information describes for example the anatomy of the human bodies and is extracted for example from medical image information about the human bodies. The atlas of a femur, for example, can comprise the head, the neck, the body, the greater trochanter, the lesser trochanter and the lower extremity as objects which together make up the complete structure. The atlas of a brain, for example, can comprise the telencephalon, the cerebellum, the diencephalon, the pons, the mesencephalon and the medulla as the objects which together make up the complex structure. One application of such an atlas is in the segmentation of medical images, in which the atlas is matched to medical image data, and the image data are compared with the matched atlas in order to assign a point (a pixel or voxel) of the image data to an object of the matched atlas, thereby segmenting the image data into objects.

### Imaging Methods

In the field of medicine, imaging methods (also called imaging modalities and/or medical imaging modalities) are used to generate image data (for example, two-dimensional or three-dimensional image data) of anatomical structures (such as soft tissues, bones, organs, etc.). The term “medical imaging methods” is understood to mean (advantageously apparatus-based) imaging methods (for example so-called medical imaging modalities and/or radiological imaging methods) such as for instance computed tomography (CT) and cone beam computed tomography (CBCT, such as volumetric CBCT), x-ray tomography, magnetic resonance tomography (MRT or MRI), conventional x-ray, sonography and/or ultrasound examinations, and positron emission tomography. For example, the medical imaging methods are performed by the analytical devices. Examples for medical imaging modalities applied by medical imaging methods are: X-ray radiography, magnetic resonance imaging, medical ultrasonography or ultrasound, endoscopy, elastography, tactile imaging, thermography, medical photography and nuclear medicine functional imaging techniques as positron emission tomography (PET) and Single-photon emission computed tomography (SPECT), as mentioned by Wikipedia. The image data thus generated is also termed “medical imaging data”. Analytical devices for example are used to generate the image data in apparatus-based imaging methods.

### Mapping

Mapping describes a transformation (for example, linear transformation) of an element (for example, a pixel or voxel), for example the position of an element, of a first data set in a first coordinate system to an element (for example, a pixel or voxel), for example the position of an element, of a second data set in a second coordinate system (which may have a basis which is different from the basis of the first coordinate system). In one embodiment, the mapping is determined by comparing (for example, matching) the color values (for example grey values) of the respective elements by means of an elastic or rigid fusion algorithm. The mapping is embodied for example by a transformation matrix (such as a matrix defining an affine transformation).

### BRIEF DESCRIPTION OF THE DRAWINGS

In the following, the invention is described with reference to the appended figures which give background explanations and represent specific embodiments of the invention. The scope of the invention is however not limited to the specific features disclosed in the context of the figures, wherein

FIG. 1 illustrates a method for identifying a region of interest according to the present disclosure that can be applied to two-dimensional or three-dimensional ultrasound image data;

FIGS. 2a and 2b illustrate methods for identifying a region of interest according to the present disclosure for two-dimensional or three-dimensional ultrasound image data, respectively;

FIG. 3 is a schematic illustration of the system according to the present disclosure;

FIGS. 4a and 4b illustrate methods for performing image registration for two-dimensional or three-dimensional ultrasound image data, respectively;

FIGS. 5a and 5b illustrate methods for performing coarse to fine image registration for two-dimensional or three-dimensional ultrasound image data, respectively; and

FIGS. 6a and 6b illustrate exemplary first and second planes of two-dimensional ultrasound image data and the region of interest in three-dimensional ultrasound image data, respectively.

### DESCRIPTION OF EMBODIMENTS

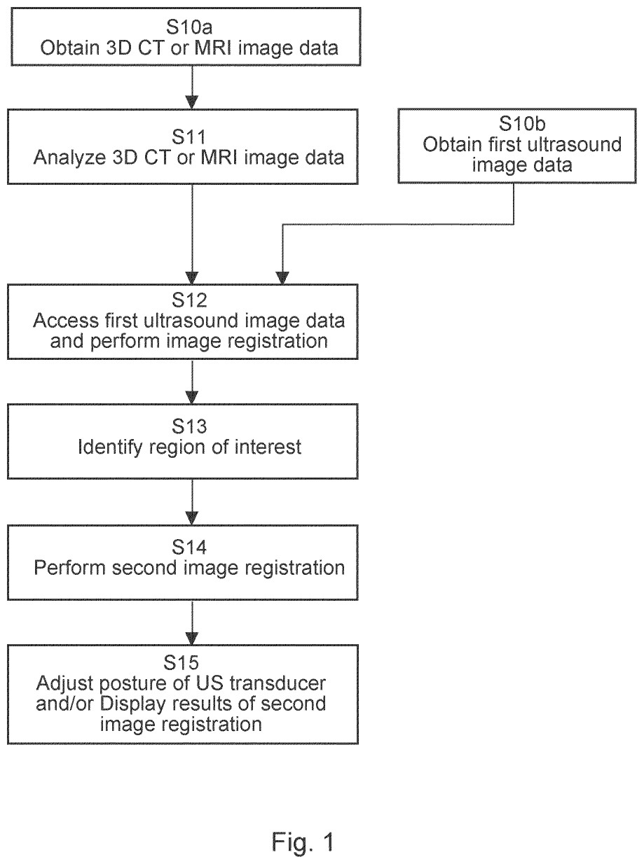

FIG. 1 illustrates the basic steps of a computer-implemented method for identifying a region of interest for image registration according to the present disclosure.

The method comprises, in step S11, analyzing three-dimensional CT or MRI image data of a tissue to identify one or more structural elements therein and determining a spatial arrangement of the structural elements. For example, one or more tubular structures like vessels may be identified. Any of the above methods may be used to do so. For example, the Eigenvalues resulting from a single value decomposition of a Hessian Matrix of the 3D image data may be used to identify presence and approximate shape of structural elements. For example, a combination of a low and two high Eigenvalues points towards a tubular structure being present.

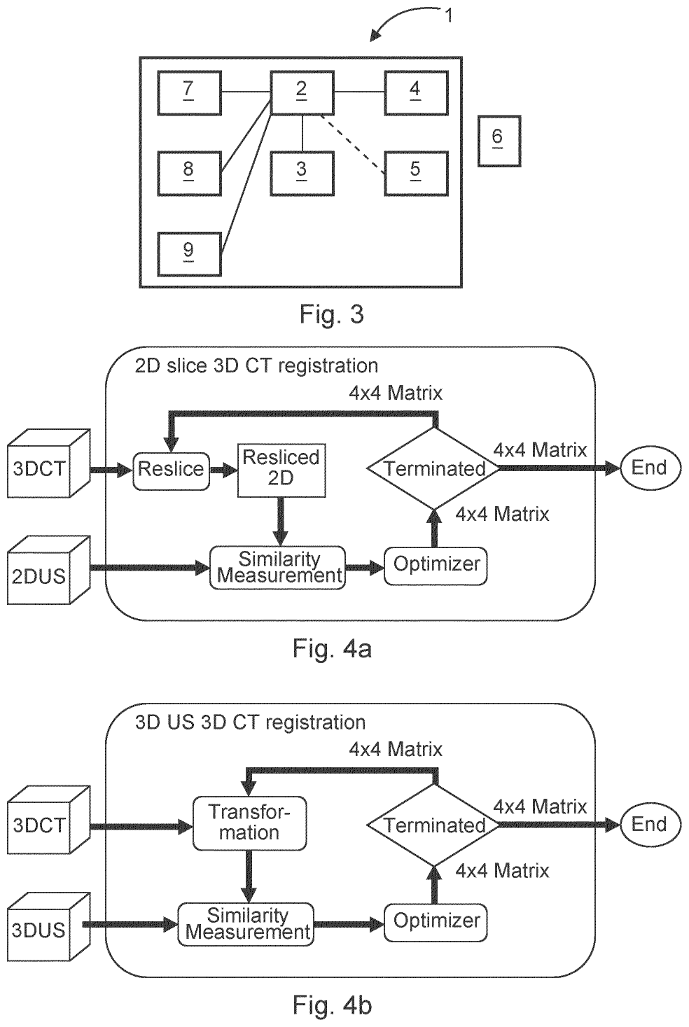

The method further comprises, in step S12, accessing first ultrasound image data of the tissue and performing a first image registration of the first ultrasound image data with the three-dimensional CT or MRI image data. The CT or MRI image data may have to undergo processing before the registration is performed, as is explained in the context of FIGS. 4a and 4b below. The registration is performed at a first accuracy. That is, a coarse image registration may be performed. This may, in particular, be achieved by performing the image registration with more weight on speed than accuracy. In particular, a low density of pixels or voxels may be used for the coarse image registration.

The first image registration may, for example, be performed by means of aligning structural elements identified in step S11. However, the present disclosure is not limited to this. any other suitable registering processing may be used.

The method further comprises, in step S13, identifying, based on the spatial arrangement of the structural elements, a region of interest for a subsequent second image registration of second ultrasound image data of the tissue with the CT or MRI image data at a second accuracy that is higher than the first accuracy. Identifying the region of interest may comprise identifying a region that has a higher number and/or density of structural elements than other regions.

The identification of the region of interest may also be performed taking into account boundary conditions, for example, that the region of interest be in a plane and/or that the region of interest has a certain size and/or shape. Furthermore, the region of interest may be identified such that a predetermined amount of computing resources and/or amount of data is expected to not be exceeded when performing a subsequent second image registration.

In an optional step S14, the second image registration of the second ultrasound image data with the three-dimensional CT or MRI image data may be performed at higher accuracy than the first image registration. The second image registration may, for example, be performed by means of aligning the structural elements identified in step S11. However, the present disclosure is not limited to this. any other suitable registering processing may be used.

In a further optional step S15, the second image registration is used for real-time adjustment of posture of an ultrasound transducer with respect to the tissue. The step may optionally comprise or be replaced with outputting a representation of the registered image data.

The method may optionally also comprise the step S10a of obtaining CT or MRI image data of the tissue using a CT or MRI scanner in advance of step S11.

The method may optionally also comprise, particularly subsequently to obtaining CT or MRI image data, obtaining the first ultrasound image data in step S10b. The timing is not particularly limited. For example, it may be entirely performed after performing step S11 or it may start already during step S11.

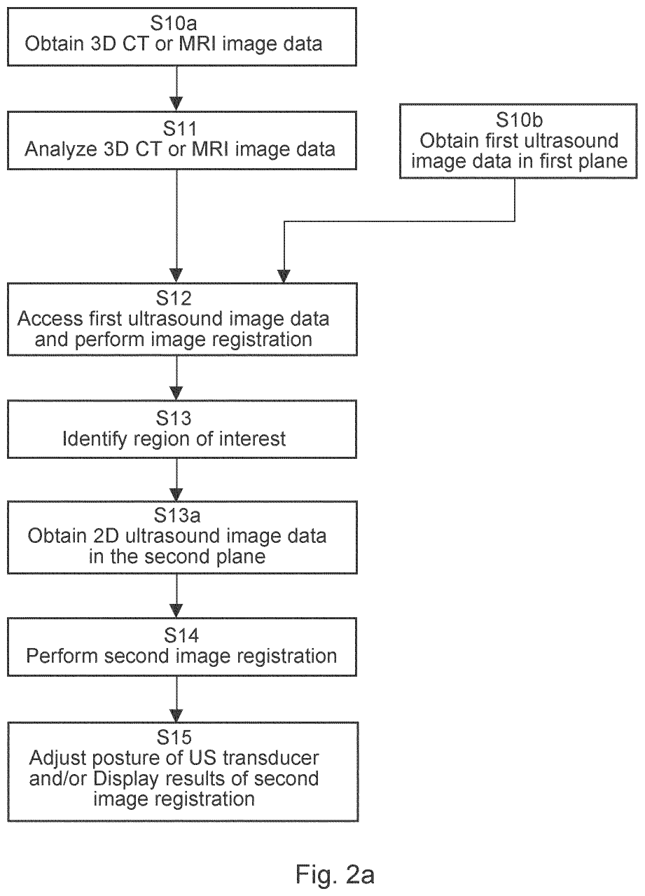

FIG. 2a illustrates the basic steps of a computer-implemented method for identifying a region of interest for image registration according to the present disclosure wherein the first ultrasound image data are two-dimensional ultrasound image data.

Steps that correspond to the steps described above in the context of FIG. 1 will not be described in detail below and reference is made to the above description.

Step S10b of obtaining first ultrasound image data may comprise obtaining two-dimensional image data in a first plane using an ultrasound transducer.

Step S12 of FIG. 1 may be performed using any suitable method for registering two-dimensional image data with three-dimensional image data. An example will be provided below in the context of FIG. 4a.

Step 13, i.e., identifying, based on the spatial arrangement of the structural elements, a region of interest in this case comprises determining a second plane that may, for example, be chosen so as to be as close to perpendicular to the first plane as possible and to comprise as many structural elements as possible.

Furthermore, the method may optionally comprise, in step S13a, performed after identifying the region of interest, obtaining two-dimensional image data in the second plane by means of the ultrasound transducer.

In optional step S14, i.e., the second image registration of the second ultrasound image data with the three-dimensional CT or MRI image data at higher accuracy than the first image registration, any method suitable for performing image registration of two-dimensional image data and three-dimensional image data may be used to register at least the image data obtained in the second plane with the there-dimensional image data.

FIG. 2b illustrates the basic steps of a computer-implemented method for identifying a region of interest for image registration according to the present disclosure wherein the first ultrasound image data are three-dimensional ultrasound image data.

Steps that correspond to the steps described above in the context of FIG. 1 will not be described in detail below and reference is made to the above description.

Step S10b of obtaining first ultrasound image data may comprise obtaining three-dimensional image data using an ultrasound transducer.

Step S12 of FIG. 1 may be performed using any suitable method for registering three-dimensional image data with three-dimensional image data. An example will be provided below in the context of FIG. 4b.

Step 13, i.e., identifying, based on the spatial arrangement of the structural elements, a region of interest in this case comprises determining a group of voxels that comprises at least part of a structural element. For example, a structural element, like a vessel, may extend, in the first ultrasound image data over various voxels, usually adjacent voxels. The region of interest may be determined as the region comprising these voxels. Any areas of the first ultrasound image data that do not comprise a structural element may be determined not to be part of the region of interest. It is noted that in some embodiments, for example when the region of interest is, for example, determined to have certain shape that differs from the shape of the structural element, for example be a cuboid, the region of interest may, nonetheless, comprise voxels that do not comprise any structural elements.

Furthermore, the method may optionally comprise, in step S13b, performed after identifying the region of interest, obtaining additional image data for the region of interest, for example by means of the ultrasound transducer.

In optional step S14, i.e., the second image registration of the second ultrasound image data with the three-dimensional CT or MRI image data at higher accuracy than the first image registration, any method suitable for performing image registration of two-dimensional image data and three-dimensional image data may be used to register at least the image data obtained in the second plane with the there-dimensional image data.

It is noted that in case of the three-dimensional ultrasound image data, the ultrasound image data used for the first image registration may have, particularly evenly distributed, voxels of a first voxel density and the image data used for the second image registration may have second voxel density, that is higher than the first voxel density, in the region of interest, and have lower voxel density, in particular the first voxel density in remaining regions of the image data. As such, the second ultrasound image data provides more detail in the region of interest than in other regions.

Thus, in optional step 14, the second image registration may have higher accuracy due to the additional information provided in the region of interest that contains structural elements that can be used for the image registration, while not requiring too much computing resources.

The image data used for the second registration may have been acquired together with the first image data, and simply have been disregarded for the first image registration. Alternatively, the data used in the second image registration may at least partially be obtained in optional step S13b.

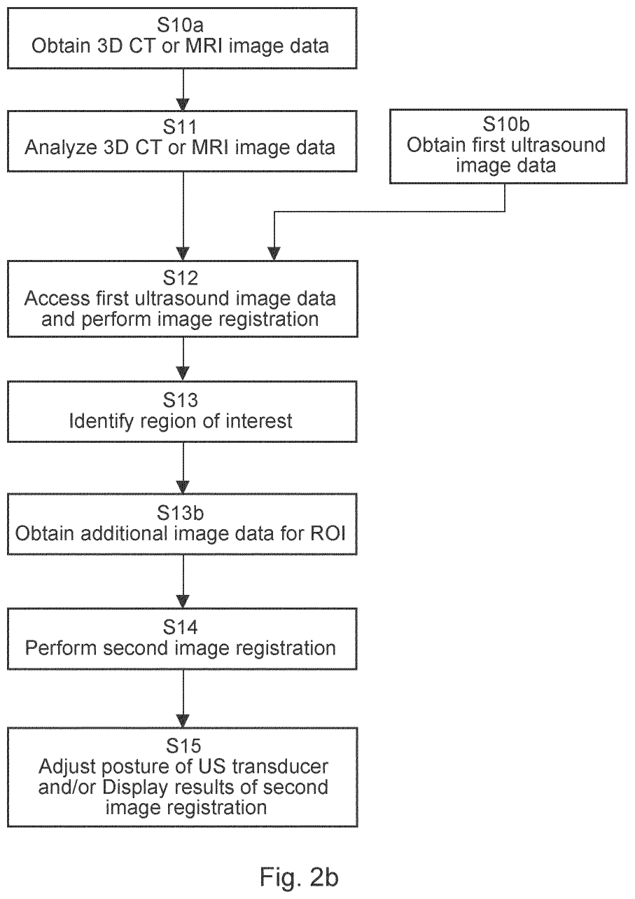

FIG. 3 is a schematic illustration of a system 1 for identifying a region of interest for image registration according to the present disclosure.

The system, in this case, may comprise a processing unit 2, which may be part of a computer, and an electronic data storage device (such as a hard disc) 3, which may also be part of a/the computer, the electronic data storage device for storing data used in the identification of the region of interest and/or the image registration. The system may optionally also comprise an ultrasound transducer 4 (such as a 2D or 3D ultrasonic transducer) configured to obtain the first ultrasound image data and/or the second ultrasound image data. The system may optionally also comprise one or more instruments 5 to be used for interacting with a tissue. A tissue 6 is shown for sake of illustration, but it is to be understood, that the tissue is not part of the system.

The system may, optionally, comprise a display device 7 and/or a user interface 8 for receiving user input, which may be external to or integrated in the display device. The system may also optionally comprise a CT or MRI scanner 9 configured to obtain the CT or MRI image data. The computer, in particular the processing unit, may be configured to perform one or more, in particular all, of the steps of the methods according to the present disclosure, particularly the method of the claims or the above-described methods.

FIG. 4a illustrates an example of how, in general, image registration may be performed in case of two-dimensional ultrasound, 2DUS, image data and three-dimensional CT, 3DCT, image data that results in 4×4 matrix.

As can be seen in the flowchart, the 3DCT image data is first resliced to obtain 2D information and then a similarity measurement is performed based on the 2D information and the 2DUS image data and input into an optimizer. The optimizer outputs a 4×4 matrix, which may be input into the reslicing step, such that the method steps are repeated, or may be output as the 4×4 matrix in which the image registration results, in which case the method may end.

FIG. 4b illustrates an example of how, in general, image registration may be performed in the case of three-dimensional ultrasound, 3DUS, image data and three-dimensional CT, 3DCT, image data.

As can be seen in the flowchart, a transformation of the 3DCT image data is performed and then a similarity measurement is performed based on the transformed data and the 3DUS image data and input into an optimizer. The optimizer outputs a 4×4 matrix, which may be input into the transformation step, such that the method steps are repeated, or may be output as the 4×4 matrix in which the image registration results, in which case the method may end.

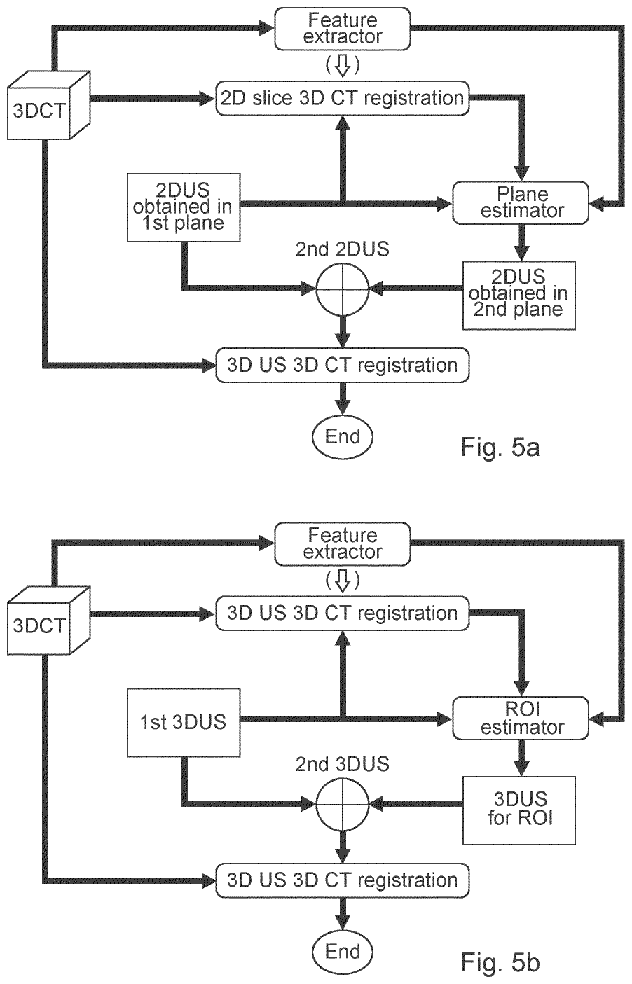

FIG. 5a illustrates a concrete example of coarse to fine image registration in the case of two-dimensional ultrasound, 2DUS, image data and three-dimensional CT, 3DCT, image data.

As can be seen in the flowchart, the 3DCT image data is input into a feature extractor, which, for example, may identify structural elements like vessels. Furthermore, the 3DCT image data and first 2DUS image data, obtained in a first plane, are used to perform a first image registration, which is a coarse image registration. Optionally (therefore the arrow is illustrated in parentheses), some of the output of the feature extractor may also be used for the first image registration. The output from the first image registration and the feature extractor are input into a plane estimator that estimates a second plane, for example in accordance with the methods described above. The second plane may be seen as a region of interest. 2DUS image data is then obtained in the second plane. In the example of FIG. 5a, the 2DUS image data obtained in the second plane is combined with the first 2DUS image data to obtain second ultrasound image data. The first 2DUS image data obtained in the first plane may only optionally be included in the second ultrasound image data (therefore the arrow is illustrated in parentheses). The second ultrasound image data and the 3DCT image data are used to perform a second image registration, which is a fine image registration. It can be performed with higher accuracy due to selecting an appropriate second plane. Then the method may end.

FIG. 5b illustrates a concrete example of coarse to fine image registration in the case of three-dimensional ultrasound, 3DUS, image data and three-dimensional CT, 3DCT, image data.

As can be seen in the flowchart, the 3DCT image data is input into a feature extractor, which, for example, may identify structural elements like vessels. Furthermore, the 3DCT image data and first 3DUS image data are used to perform a first image registration, which is a coarse image registration. Optionally (therefore the arrow is illustrated in parentheses), some of the output of the feature extractor may also be used for the first image registration. The output from the first image registration and the feature extractor are input into a ROI (region of interest) estimator that estimates the region of interest, for example in accordance with the methods described above. Additional image data, e.g., additional voxels, for the region of interest is combined with first 3DUS image data to obtain second 3DUS image data in the example of FIG. 5b. The first 3DUS image data may only optionally be included in the second ultrasound image data (therefore the arrow is illustrated in parentheses). The second ultrasound image data and the 3DCT image data are used to perform a second image registration, which is a fine image registration. It can be performed with higher accuracy due to having more information in the region of interest, for example a higher voxel density. Then the method may end.

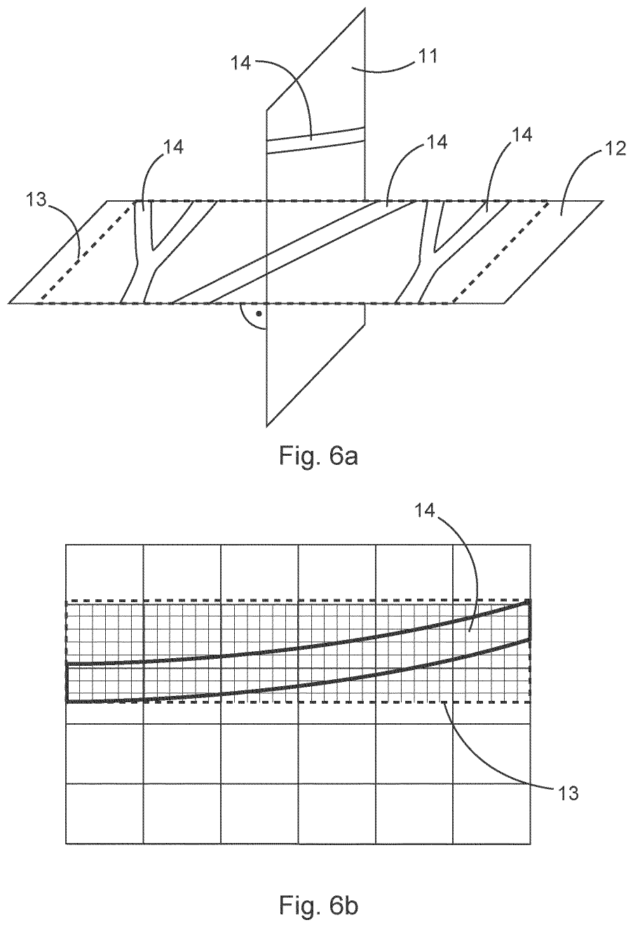

FIG. 6a illustrates an exemplary first plane 11 and second plane 12, which may be or comprise a region of interest 13, of the methods of the present disclosure wherein two-dimensional ultrasound image data is registered with the three-dimensional CT or MRI image data. Structural elements 14, for example vessels, are depicted schematically.

FIG. 6b illustrates an exemplary region of interest (ROI) 13 in three-dimensional ultrasound image data, wherein a structural element 14, for example a vessel, is depicted in the ROI. The grid that is shown in FIG. 6b illustrates that the voxel-density in the ROI is higher than in other regions for the second image registration. In this example, the ROI is shaped as a rectangle merely for sake of illustration.

While the invention has been illustrated and described in detail in the drawings and foregoing description, such illustration and description are to be considered exemplary and not restrictive. The invention is not limited to the disclosed embodiments. In view of the foregoing description and drawings it will be evident to a person skilled in the art that various modifications may be made within the scope of the invention, as defined by the claims.

1. A computer-implemented method for identifying a region of interest for image registration, the method comprising:
analyzing three-dimensional CT or MRI image data of a tissue to identify one or more structural elements therein and determining a spatial arrangement of the structural elements;
accessing first ultrasound image data of the tissue and performing a first image registration of the first ultrasound image data with the CT or MRI image data at a first accuracy; and
identifying, based on the spatial arrangement of the structural elements, a region of interest for a subsequent second image registration of second ultrasound image data of the tissue with the CT or MRI image data at a second accuracy that is higher than the first accuracy.

2. The method according to claim 1, wherein identifying the region of interest based on the spatial arrangement of the structural elements comprises determining the number and/or density of the structural elements of different areas in the CT or MRI image data and/or determining areas in the CT or MRI image data that do not have any structural elements.

3. The method according to claim 1, wherein identifying the region of interest based on the spatial arrangement of the structural elements is performed taking into account boundary conditions as to the region of interest.

4. The method according to claim 1, wherein performing the first image registration comprises aligning at least some of the structural elements identified in the CT or MRI image data with corresponding structural elements in the first ultrasound image data, and/or wherein performing the second image registration comprises aligning at least some of the structural elements identified in the CT or MRI image data in the region of interest with corresponding structural elements in the second ultrasound image data.

5. The method according to claim 1, wherein identifying one or more structural elements comprises determining high order image derivatives in the CT or MRI image data and determining, based thereon, the presence and approximate shape and/or orientation of the one or more structural elements.

6. The method according to claim 1, wherein identifying the structural elements comprises obtaining Eigenvalues of a Hessian matrix applied to the CT or MRI image data and, based on the Eigenvalues, identifying the structural elements.

7. The method according to claim 6, wherein identifying the structural elements comprises categorizing each of the Eigenvalues as low or as high positive or as high negative and determining that a structural element is present when a predetermined combination of categories of the Eigenvalues is determined.

8. The method according to claim 1, wherein the first ultrasound image data and the second ultrasound image data is three-dimensional ultrasound image data, wherein the first ultrasound image data comprises a first subset of voxels of ultrasound image data of the tissue, and wherein the second ultrasound image data comprises a second subset of voxels of ultrasound image data of the tissue, the second subset of voxels having a higher voxel-density in the region of interest than the first subset of voxels.

9. The method according to claim 1, wherein the first ultrasound image data and the second ultrasound image data is three-dimensional ultrasound image data, and wherein identifying the region of interest comprises identifying only areas wherein at least one of the structural elements is present as being part of the region of interest and/or wherein the second ultrasound image data provides more detail in the region of interest than the first image data, and/or wherein the second ultrasound image data is non-uniform, providing more detail in the region of interest than in other regions.

10. The method according to claim 1, wherein the first ultrasound image data is two-dimensional ultrasound image data obtained in a first plane and the second ultrasound image data is two-dimensional image data to be obtained in a second plane, and wherein determining the region of interest comprises determining the second plane by performing an optimization procedure that optimizes for the first plane and the second plane being close to perpendicular to each other and for the second plane comprising a high number and/or density of the structural elements.

11. The method according to claim 1, further comprising performing the second image registration of the second ultrasound image data with the CT or MRI image data at the second accuracy.

12. The method according to claim 1, wherein the first ultrasound image data is ultrasound image data obtained at a later time than the CT or MRI image data and/or wherein the second ultrasound image data comprises at least part of the first ultrasound image data, and/or wherein the second ultrasound image data is ultrasound image data obtained after identifying the region of interest and/or wherein the second ultrasound image data is ultrasound image data obtained before identifying the region of interest.

13. The method of according to claim 1, wherein the second image registration is used for real-time adjustment of posture of an ultrasound transducer with respect to the tissue.

14. The method according to claim 1, further comprising obtaining CT or MRI image data of the tissue using a CT or MRI scanner and/or obtaining the first ultrasound image data and/or the second ultrasound image data using a two-dimensional or a three-dimensional ultrasound transducer.

15. A system for identifying a region of interest for image registration, the system comprising:
a processing unit operable to:
analyze three-dimensional CT or MRI image data of a tissue to identify one or more structural elements therein and determining a spatial arrangement of the structural elements;
access first ultrasound image data of the tissue and performing a first image registration of the first ultrasound image data with the CT or MRI image data at a first accuracy; and
identify, based on the spatial arrangement of the structural elements, a region of interest for a subsequent second image registration of second ultrasound image data of the tissue with the CT or MRI image data at a second accuracy that is higher than the first accuracy.

16. The system according to claim 15, further comprising a CT or MRI scanner configured to obtain the CT or MRI image data and/or an ultrasound transducer configured to obtain the first ultrasound image data and/or the second ultrasound image data.

17. (canceled)

18. A non-transitory computer-readable storage medium storing a program for identifying a region of interest for image registration, that when executed on at least one processing unit of a computer or loaded onto the at least oen processing unit of the computer, causes the computer to:
analyze three-dimensional CT or MRI image data of a tissue to identify one or more structural elements therein and determining a spatial arrangement of the structural elements;
access first ultrasound image data of the tissue and performing a first image registration of the first ultrasound image data with the CT or MRI image data at a first accuracy; and
identify, based on the spatial arrangement of the structural elements, a region of interest for a subsequent second image registration of second ultrasound image data of the tissue with the CT or MRI image data at a second accuracy that is higher than the first accuracy.
